(19) United States
(12) Patent Application Publication
(10) Pub. No.: US 2005/0054594 A1
(43) Pub. Date: Mar. 10, 2005

(54) Methods for particle-assisted polynucleotide immunization using a pulsed electric field

(76) Inventors: Lei Zhang, San Diego, CA (US); Georg Widera, Mountain View, CA (US); Dietmar P. Rabussay, San Diego, CA (US)

Correspondence Address: BIOTECHNOLOGY LAW GROUP, 658 MARSOLAN AVENUE, SOLANA BEACH, CA, 92075, US

(21) Appl. No.: 10/498,923

(22) PCT Filed: Dec. 16, 2002
(86) PCT No.: PCT/US02/40467

(30) Foreign Application Priority Data
Dec. 14, 2001 (US) 60340784

(51) Int. Cl. A61K 48/00; A61N 1/30

(52) U.S. Cl. 514/44; 604/20

(57) ABSTRACT

Methods are provided for enhancing an immune response induced by administration of a DNA vaccine. In the invention methods a DNA vaccine encoding an antigen and non-chemically associated adjuvant particles are injected into muscle, dermal or mucosal tissue of a subject at substantially the same time and the tissue is subjected to a pulsed electric field of sufficient strength to result in the DNA vaccine entering cells of the target tissue. The immune response to the antigen is enhanced as compared to when the DNA vaccine is administered alone or in combination with either of the electric pulses or the adjuvant particles without the other.

The present invention relates generally to methods and compositions for generating an immune response in a subject. In particular, the invention relates to the use of electrically, assisted delivery of polynucleotides encoding an antigen for the purpose of generating an immune response in a subject.

### BACKGROUND OF THE INVENTION

Numerous vaccine formulations that include attenuated pathogens or subunit protein antigens have been developed. Conventional vaccine compositions often include immunological adjuvants to enhance immune responses. For example, depot adjuvants are frequently used which adsorb and/or precipitate administered antigens and which can retain the antigen at the injection site. Typical depot adjuvants include aluminum compounds and water-in-oil emulsions. However, depot adjuvants, although increasing antigenicity, often provoke severe persistent local reactions, such as granulomas, abscesses and scarring, when injected subcutaneously or intramuscularly. Other adjuvants, such as lipopolysacharrides, can elicit pyrogenic responses upon injection and/or Reiter's symptoms (influenza-like symptoms, generalized joint discomfort and sometimes anterior uveitis, arthritis and urethritis). Saponins, such as Quillaja sappnaria, have also been used as immunological adjuvants in vaccine compositions against a variety of diseases.

More particularly, Complete Freund's adjuvant (CFA) is a powerful immunostimulatory agent that has been successfully used with many antigens on an experimental basis. CFA includes three components: a mineral oil, an emulsifying agent, and killed mycobacteria, such as Mycobacterium tuberculosis. Aqueous antigen solutions are mixed with these components to create a water-in-oil emulsion. Although effective as an adjuvant, CFA causes severe side effects primarily due to the presence of the mycobacterial component, including pain, abscess formation and fever. CFA, therefore, is not used in human and veterinary vaccines.

Despite the presence of such adjuvants, conventional vaccines often fail to provide adequate protection against the targeted pathogen. In this regard, there is growing evidence that vaccination against intracellular pathogens, such as a number of viruses, should target both the cellular and humoral arms of the immune system.

More particularly, cytotoxic T-lymphocytes (CTLs) play an important role in cell-mediated immune defense against intracellular pathogens such as viruses and tumor-specific antigens produced by malignant cells. CTLs mediate cytotoxicity of virally infected cells by recognizing viral determinants in conjunction with class I MHC molecules displayed by the infected cells. Cytoplasmic expression of proteins is a prerequisite for class I MHC processing, and presentation of antigenic peptides to CTLs. However, immunization with killed or attenuated viruses often fails to produce the CTLs necessary to curb intracellular infection. Furthermore, conventional vaccination techniques against viruses displaying marked genetic heterogeneity and/or rapid mutation rates that facilitate selection of immune escape variants, such as HIV or influenza are problematic. Accordingly, alternative techniques for vaccination have been developed.

Particulate carriers with adsorbed or entrapped antigens have been used in an attempt to elicit adequate immune responses. Such carriers usually present multiple copies of a selected antigen to the immune system and promote trapping and retention of antigens in local lymph nodes. The particles can be phagocytosed by macrophages and can enhance antigen presentation through cytokine release. Examples of particulate carriers include metallic particles and those derived from various polymers, such as polymethyl methacrylate polymers, as well as-particles derived from poly(lactides), and poly(lactide-co-glycolides), known as PLG. Polymethyl methacrylate polymers are nondegradable while PLG particles biodegrade by random nonenzymatic hydrolysis of ester bonds to lactic and glycolic acids that are excreted along normal metabolic pathways.

Recent studies have shown that PLG particles with entrapped antigens are able to elicit cell-mediated immunity and/or mucosal IgA responses when administered orally. Additionally, both antibody and T-cell-responses have been-induced in mice vaccinated with a PLG-entrapped Mycobacterium tuberculosis. Antigen-specific CTL responses have also been induced in mice using a microencapsulated short synthetic peptide.

Another recent development with regard to vaccines is the administration to a subject of a polynucleotide that encodes an antigen for production of the desired antigen in vivo by the subject. Such “DNA vaccines” can be administered as “naked” DNA or in a carrier formulation, adsorbed to or otherwise chemically, associated with (or within) the surface of particles, contained within an expression vector or plasmid, and the like, and by such routes of administration as mucosal exposure, injection-into tissue, usually muscle, and the like.

It is also known to utilize various forms of electric a impulses applied to skin or other tissue, such as muscle, via various types of electrodes as a means to deliver a drug, nucleic acid, or immunogenic-agent to a subject. For example, by selection of the appropriate electrical parameters, electroporation of cells in tissue to which a DNA vaccine or other type of immune-inducing agent is applied or injected can be used to enhance delivery of the vaccine to the subject for the purpose of raising a protective immune response.

However, there is a need in the art for new and better methods for delivery of antigen-encoding polynucleotides for raising a protective immune response in subjects. For this purpose, the co-administration of an adjuvant of biodegradable or inert particles and a pulsed electric field at the target tissue, wherein the particles and polynucleotide are not substantially chemically associated with each other, has not heretofore been described.

### SUMMARY OF THE INVENTION

The present invention is based on the surprising and unexpected discovery that the immune response of a subject to a DNA vaccine administered into skin, muscle or mucosa can be enhanced by co-administering an adjuvant of biodegradable or inert particles and a pulsed electric field at the target tissue, wherein the particles and polynucleotide are not substantially chemically associated with each other. The use of such combinations provides a safe and effective approach for enhancing the immunogenicity of a wide variety of antigens.

Accordingly, in one embodiment, the invention provides methods for inducing an immune response by administration of an antigen-encoding polynucleotide to a subject. In the invention methods, an immunogenic-effective amount of at least one polynucleotide encoding an antigen is introduced into a target tissue of a subject by at route selected from the group consisting of, intramuscularly intradermally, subcutaneously and intramucosally; generating a pulsed electric field at the target tissue of sufficient strength and at substantially the same time as the introduction of the polynucleotide so as to result in the polynucleotide entering cells of the target tissue for expression therein and so as to result in generation in the subject of an immune response to the antigen encoded by the polynucleotide; and introducing an adjuvant-effective quantity of particles into the target tissue within several days of the introduction of the polynucleotide and the generation, of the electric field, wherein the polynucleotide and the particles are not substantially chemically associated with one another prior to the introducing thereof. By this method, an enhanced immune response, as compared with the immune response resulting from other modes of immunization involving administration of such a polynucleotide encoding the antigen, is achieved.

In another embodiment, the invention provides methods for inducing an immune response by administration of antigen-encoding polynucleotide to a subject by introducing an immunogenic-effective amount of at least one polynucleotide encoding an antigen into a target tissue of a subject by intramuscular injection; generating a pulsed electric field at the target tissue of sufficient strength and at substantially the same time as the introduction of the polynucleotide so as to result in the polynucleotide entering cells of the target tissue for expression therein and so as to result in generation in the subject of an immune response to the antigen encoded by the polynucleotide; and introducing adjuvant-effective quantity of particles into the target tissue within several days of the introduction of the polynucleotide and the generation of the electric field, wherein the polynucleotide and the particles are not substantially chemically associated with one another prior to the introducing thereof. The immune response resulting from the invention methods is enhanced as compared with an immune response resulting from other modes of immunization involving administration of such a polynucleotide encoding the antigen.

These and other embodiments of the present invention will readily occur to those of ordinary skill in the art in view of the disclosure herein.

### BRIEF DESCRIPTION OF THE FIGURES

FIG. 1 is a graph showing the results of comparatives tests conducted to measure secreted embryonic alkaline phosphatase (SEAP) gene expression in hairless mice when DNA was injected into tibialis muscle in the following combinations: Together with gold particles and electroporation (column 1); together with gold particles and no electroporation (column 2), together with electroporation and no particles (column 3), or DNA alone (column 4). □=gene expression on day 0; ▪=gene expression on day 3 post injection; the column with slanted stripes=gene expression 7 days post injection. In this example, “together with gold particles” means that the DNA and the particles were not substantially chemically associated with each other.

### DETAILED DESCRIPTION OF THEE INVENTION

The practice of the present invention will employ, unless otherwise indicated, conventional methods of chemistry, biochemistry, molecular biology, immunology and pharmacology, within the skill of the art. Such techniques are explained filly in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir, and C. C. Blackwell, eds., 1986, Blackwell Scientific Publications); and Sambrook and Russell., Molecular Cloning: A Laboratory Manual (3rd Edition, 2000).

All publications, patents and patent applications cited herein are hereby; incorporated by reference in their entirety.

As used in this specification and in the appended claims, the singular forms “a,” “an” and “the” include plural references unless the content, clearly dictates otherwise.

In describing the present invention, the following terms will be employed, and are intended to be defined as indicated below.

By “inert” is meant a stable composition that will not, on its own, react chemically with a living body in any appreciable manner when introduced into a body.

By “polynucleotide” is meant nucleic acid polymers, such as DNA, cDNA, mRNA and RNA, which can be linear, relaxed circular, supercoiled or condensed and single or double stranded. The polynucleotide can also contain one, or more, moieties that are chemically modified, as compared to the naturally occurring moiety. The polynucleotide can be provided without placement into a delivery vehicle (e.g., as a “naked” polynucleotide), in an expression plasmid or other suitable type of vector, such as is known in the art. It is specifically contemplated as within the scope of the invention that the polynucleotide can be an oligonucleotide. In addition to the polynucleotide being administered in “naked” form, the polynucleotide may also be administered in a formulated form or modified form. For example, the polynucleotide may be formulated by mixing it with a protective, interactive, non-condensing (PINC) polymer Newell, J. G., et al., Gene therapy for the treatment of hemophilia B using: PINC-formulated plasmid delivered to muscle with electroporation. Molecular Therapy, 3:574-583 (2000)) or the polynucleotide can be modified by attaching a peptide or other chemical entity, such as a marker molecule, to the polynucleotide (Zelphati, O., et al., PNA-dependent gene chemistry: stable coupling of peptides and oligonucleotides to plasmid DNA [Biotechniques 28:304-310; 312-314; 316 (2000)).

By “chemically associated with” is meant chemically complexed with, chemically attached to, coated with or on, adsorbed to, or otherwise chemically associated. For instance, nucleic acid that is coated on or adsorbed, to particles is chemically associated with the particles. Association can be by covalent or non-covalent bonds. In the context of the present invention, the particles are not “chemically associated with” the polynucleotide encoding the antigen of interest or with a delivery vehicle for the polynucleotide, such as a plasmid or vector containing the polynucleotide. Thus, the particles and the polynucleotide or polynucleotide-containing plasmid or vector are not, to any significant extent, adsorbed onto one another, bound or bonded together or associated in a complex. Instead, the polynucleotide or the polynucleotide-containing plasmid or vector remain substantially separate and distinct from the particles, even when present in the same solution, suspension or carrier. One can determine that the particles and polynucleotide are not substantially chemically associated with each other by a variety of means known to those of skill in this art. For example, a sample of a solution of polynucleotide and particle prepared for administration to a subject could be separated into particles and polynucleotide by centrifugation and levels of association could be shown by gel electrophoresis. Or, the sample could be run on a gel and the lack of chemical association could be thereby detected. Furthermore, the DNA vaccines are in solution, generally 1×PBS saline, or water, which also prevents the chemical association of DNA and particles.

By “dermal tissue” is meant epidermis and dermis below the stratum corneum.

By “antigen presenting cells” or “APCs” is meant monocytes, macrophages, dendritic cells, Langerhans cells, and the like, which initiate cellular processes allowing the APC to sequester antigen and present the antigen, or a portion thereof, to T cells after migration to draining lymph nodes.

By “intradermal” and “intradermally” is meant administration into, but not on the surface of, dermal layers of the skin. For example, an intradermal route includes, but is not limited to, tumors of dermal cells.

By “intramuscular administration” and “intramuscularly” is meant administration into the substance of the muscle, i.e., into the muscle bed.

By “intramucosal administration” and “intramucosally” is meant administration into the mucosa or mucous tissue lining various tubular structures, including but not limited to epithelium, lamina propria and, in the digestive tract, a layer of smooth muscle.

By “subcutaneous administration” and “subcutaneously” is meant administration into tissue underlying the skin.

By “immunization” is meant the process by which an individual is rendered immune or develops an immune response.

By “antibody” is meant an immune or protective protein evoked in animals, including humans, by an antigen and characterized by a specific reaction of the immune protein with the antigen.

By “at substantially the same time” with reference to the timing of the coadministration of the polynucleotide and the pulsed electric field, is meant simultaneously, or within about minutes to hours to days of a administration of each other. The particles can be administered within several days either before or after administration of the polynucleotide and the pulsed electric field. For example, in one preferred embodiment, polynucleotide is introduced first, followed by application of the pulsed electric field and introduction of particles, together or sequentially, at a time or, times up to about 3 hours after introduction. In another embodiment, introduction of polynucleotide and application of the pulsed electric field, is together or sequentially within a few hours of one another and the particles are introduced at a time or times up to about 3 days, for example up to two days, or up to one day, before or after introduction of the particles and electroporation. A further embodiment is the introduction of a mixture of particles and polynucleotide, wherein the particles and polynucleotide are not chemically associated with each other, and wherein the pulsed electrical field is applied at a time up to about 5 hours after introduction of the particles and formulated or unformulated (i.e., “naked”) polynucleotide. Presently preferred embodiments are those wherein the administration of polynucleotide, particle and electric pulse(s) are simultaneous or within no more than 5 minutes of each other. One of skill can determine the optimal order of introduction of the particles and polynucleotide and application of the electric field through performance of several straightforward experiments in which the timing and order of each component is varied, such as known to those of skill and set forth in Example 5.

By “antigen” is meant a molecule that contains one or more epitopes that will stimulate a host's immune system to make a humoral antibody response, or cellular antigen-specific immune response when the antigen is presented. Normally, an epitope will include between about 3-15, generally about 5-15, amino acids. For purposes of the present invention, antigens can be derived from any of several known viruses, bacteria, parasites and fungi. The term also is intended to encompass any of the various tumor antigens. Furthermore, for purposes of the present invention, an “antigen” includes those with modifications, such as deletions, additions and substitutions (generally conservative in nature), to the native sequence, so long as the protein, polypeptide or polysaccharide maintains the ability to elicit an immunological response. These modifications may be deliberate, as through site-directed mutagenesis, or may be accidental, such as through mutations of hosts that produce the antigens.

An “immune response” to an antigen composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major: histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-ells and/or other white blood cells, including those derived from CD4+ and CD8+ T-cells.

The term “particle” as used herein, refers to particles of an inert and/or biodegradable material or composition, wherein the particles have sufficient rigidity to be internalized by antigen presenting cells and can optionally have a neutral or negative charge. A particle can be solid or semi-solid. The particles will have a largest mean dimension in the range from about 0.05 micron to about 20 microns, and preferably in the range from about 0.1 micron to about 3 microns in diameter. Particles in the preferred size range can readily be internalized by antigen presenting cells. Preferred particles are microparticles, such as those, derived from noble metals, especially particulate gold as well as particulate aluminum, titanium, tungsten, and carbon. Although pure metal particles are preferred, especially pure gold particles, alloys containing from 99.5% to 95% by volume of such metals can also be used in practice of the invention methods. Such particulate metals are readily available from commercial vendors. Examples of other particle materials are liposomes, other vesicles, polymers, and the like.

An invention method “enhances immunogenicity” of the polynucleotide encoding an antigen when it hastens the appearance of an immune response (i.e., enhances kinetics of the immune response) or possesses a greater capacity to elicit an immune response than the immune response elicited by an equivalent amount of the polynucleotide without the particle/pulsed electric field adjuvant effect. Thus, the method for inducing an immune response may display “enhanced immunogenicity” because the antigen produced is more strongly immunogenic or because a lower dose of polynucleotide encoding the antigen is necessary to achieve an immune response in the subject to which it is administered, or because an efficient immune response, e.g., as manifested by, but not limited to antibody titer, is reached more rapidly after administration. In the present invention, the enhanced immune response preferably includes the advantage that the kinetics of the immune response is faster as evidenced by faster appearance of an immune response, e.g., as evidenced by a rise in antibody titer, than in other immunization protocols. Such enhanced immunogenicity can be determined by administering the polynucleotide composition and pulsed electric field, or the polynucleotide and the particles as controls to animals and comparing immune response against the invention methods using standard assays such as radioimmunoassay and ELISAs, as is well known in the art and as illustrated in the Examples herein with ELISAs.

The term “adjuvant-effective quantity” as applied to the particles used in the invention methods refers to sufficient quantity of the particles to provide the adjuvant effect for the desired immunological response and corresponding therapeutic effect. The exact amount required will vary from subject to subject, depending on the species, age, and general condition of the subject, the severity of the condition being treated, and the particular polynucleotide encoding the antigen of interest, mode of administration, e.g. whether to muscle or skin, the size and type of the particles, and the like. An appropriate “effective” amount in any individual case may be determined by one of ordinary skill in the art using routine experimentation.

The compositions comprising the polynucleotide encoding an antigen will comprise an “immunogenic-effective amount” of the polynucleotide of interest. That is, an amount of polynucleotide will be included in the compositions that, when the encoded antigen is produced in the subject, in combination with the particles and the pulsed electric field, will cause the subject to produce a sufficient immunological response in order to prevent, reduce or eliminate symptoms. An appropriate effective amount can be readily determined by one of skill in the art. Thus, an “immunogenic-effective amount” will fall in a relatively broad range that can be determined through routine trials.

As used herein, “inducing an immune response” refers to any of (i) the prevention of infection or reinfection, as in a traditional vaccine, (ii) he reduction elimination of symptoms, and (iii) the substantial or complete elimination of the pathogen in question. Thus, the methods for inducing an immune response may be effected prophylactically (prior to infection) or therapeutically (following infection).

By “pharmaceutically acceptable” or “pharmacologically acceptable” is meant a material which is not biologically or otherwise undesirable i.e., the material may be administered to an individual along with the particle adjuvant formulations without causing any undesirable biological effects or, interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

By “subject” is meant any mammal, including, without limitation, humans and other primates, including non-human primates such as chimpanzees and other apes and, monkey species; farm animals such as cattle, sheep pigs, goats and horses; domestic mammals such as dogs and cats; laboratory animals including rodents: such as mice, rats and guinea pigs, domestic pets, farm animals, such as chickens, and the like. The term does not denote a particular age. Thus, both adult and newborn individuals are included among the subjects who can be treated according to the invention methods. The invention methods described herein are intended for use in any of the above mammalian species, since the immune systems of all of these mammals operate similarly.

An invention method that elicits a cellular immune response may serve to sensitize a mammalian subject by the presentation of antigen in association with MHC molecules at the cell surface. The cell-mediated immune response is directed at cells presenting antigen at their surface. In addition, antigen-specific cytotoxic T-lymphocytes (CTLs) can be generated to allow for the future protection of an immunized host.

The ability of a particular invention method to stimulate a cell-mediated immunological response may be determined by a number of assays, such as by lymphoproliferation (lymphocyte activation) assays, CTL cell assays, or by otherwise assaying for T-lymphocytes specific for the antigen in a sensitized subject. Such assays are well known in the art. See e.g., Erickson et al., J. Immuno. (1993)151:4189-4199; Doe et al., Eur. J. Immunol. (1994)24:2369-2376; and the examples below.

Thus, an immunological response as used herein may be one which stimulates the production of CTLs, and/or the production or activation of helper T-cells. The antigen of interest may also elicit an antibody-mediated immune response. Hence, an immunological response may include one or more of the following effects: the production of antibodies by B-cells and/or the activation of suppressor T-cells. These responses may serve to neutralize infectivity; and/or mediate antibody-complement, or antibody dependent cell cytotoxicity (ADCC) to provide protection to an immunized host, e.g. against challenge by the disease causing organism or tumor cell. Such responses can be determined using standard immunoassays and neutralization assays, well known in the art.

Modes of Carrying Out the Invention

The present invention is based on the discovery that, when adjuvant particles that are not chemically associated with a DNA vaccine, are administered into a tissue with the DNA vaccine and in combination with the generation of a pulsed electric field at the tissue, an immune response to the encoded antigen is reliably generated in a subject. The invention methods provide the additional advantage that an enhanced immune response, e.g., a more rapid immune response, is achieved in a subject as compared with other types of immunization protocols tested. In some cases, as shown by the results of Example 2 below, a synergistic effect is seen such that the immune response achieved using the invention methods is greater (e.g., as measured by titer) than the additive enhanced effects that result when either the adjuvant particles or the pulsed electric field is used alone with the polynucleotide vaccine. When such a synergistic effect is seen, it is generally present at about six weeks after the initial vaccination protocol is administered at, which time a higher titer of antibody is seen in subjects treated with the invention as compared with titers in subjects treated by the other means.

Although, the individual components of the invention methods described herein were known, it was unexpected and surprising such a combination would enhance the immunogenicity of antigens produced in vivo beyond that achieved when the components were used separately or in any combination other than as recited in the invention three-part protocol.

An enhanced immune response is advantageous under many different circumstances. For instance, when protective immunization is needed quickly, such as when military troops are deployed to foreign grounds in times of emergency or when outbreaks of pathogens (e.g. anthrax) occur unexpectedly, the shorter time to reach protective immunity offered by the present invention is an advantage. Similarly, when protective immunity is quickly needed to address an acute condition or outbreak, the enhanced immunity of the present invention can address that need, as well.

The methods of the invention provide generation of a pulsed electric field in the target tissue at substantially the same time as the introduction of the polynucleotide and the particles into the tissue, wherein the electric pulses are of sufficient strength to result in the polynucleotide vaccine entering cells of the target tissue, as well as disturbing the tissue in a manner that attracts APCs and other relevant cells of the immune system. The pulsed electric field is of strength sufficient to cause electrotransport of the polynucleotide into cells of the target tissue.

One type of electrotransport is electroporation. For example, to cause electroporation of cells in muscle tissue, the pulsed electric field used in the invention methods will have low nominal electric field strength from about 50 V/cm to about 400 V/cm, preferably about 100 V/cm to about 200 V/cm. The length of pulses used in the pulsed electric field delivered to muscle will be in the range from about 1-100 milliseconds (msec), preferably 20-60 msec and about 1-6 pulses will be applied. The waveform of the electric pulses can be monopolar or bipolar. For the invention method of delivering DNA vaccines into skin, the pulsed electric field will be developed with from 1 to about 12 pulses of 50V to 80 Volts each, lasting from about 100 microseconds to 100 msec each. An alternate protocol for generating a suitable electric field in skin is to apply to the dermal tissue a short, single high voltage pulse, for example about 70V to about 100V for several hundred microseconds of duration, to break down the stratum corneum, followed by 1 to about 3 low voltage, long pulses (for example, 50 V to about 80 V for 1-100 msec) to drive the DNA vaccine into cells.

Electroporation used in performance of the invention methods can employ any type of suitable electrode as is known in the art. For example, for generation of an electric field in muscle at substantially the same time as introduction of a DNA vaccine and particles, needle electrodes comprised of two, four, or six, electrodes are preferred. Electrodes configured into pairs, opposed pairs, parallel rows, triangles, rectangles, squares, or any other suitable geometry are contemplated. In addition to invasive electrodes, an electric field can be generated in muscle by application of noninvasive or minimally invasive electrodes to skin over the site of DNA and particle delivery. For generation of an electric field in skin at substantially the same time as introduction of a DNA vaccine and particles, various invasive electrodes or noninvasive electrodes can be used. Noninvasive electrodes such as caliper electrodes, meander electrode, micropatch electrodes and micro-needle electrodes, and variations of same, are preferred. Such electrodes are commercially available and are fully described in the art. For electroporation applied to the surface of the skin, non-invasive electrodes, such as meander electrodes, or short needle electrodes of up to several millimeters in length so as to penetrate the stratum corneum are preferred. By contrast, for electroporation applied to muscle, longer needle electrodes are preferred.

Several presently preferred conditions for providing electroporation in practice of the invention methods are provided in Table 1 below:

TABLE 1

| | | | Number | | | |
|---|---|---|---|---|---|---|
| Site of | Type of | Field | of | | Applied | Frequency |
| delivery | Electrode | Strength | pulses | Pulse length | Voltage | In Hz |
| Muscle | 2-needle | Low | 1-3 | Long | N/A | 0.1-10 |
| | electrode | 150-200 V/cm | identical | 60 msec | | |
| | | | pulses | | | |
| Muscle | 4 needle | Low | 1-3 | Long | N/A | 0.1-10 |
| | electrode | 150-200 V/cm | identical | 60 msec | | |
| | | | pulses | | | |
| Muscle | 6 needle | Low | 6 | Long | N/A | 0.1-10 |
| | electrode | 100-200 V/cm | identical | 20-60 msec; | | |
| | | | pulses w/ | | | |
| | | | polarity | | | |
| | | | reversal | | | |
| Into Skin | Meander | N/A | 1-12 | Long | 50-80 V | 0.1-50 |
| Cells | | | identical | 10-100 msec | | |
| | | | pulses | | | |
| Into Skin | Micropatch | N/A | 1-6 | Long | 50-80 V | 1-50 |
| Cells | | | identical | 10-100 msec | | |
| | | | pulses | | | |
| Into Skin | Short | Low | 1-6 | Long | | 0.1-50 |
| Cells | needle | 100-250 V/cm | identical | 100 μsec-60 msec | | |
| | | | pulses | | | |

The methods of the present invention can be practiced with mucosal tissues as the target tissues, such as buccal and nasal membranes. The parameters for application of the electric charge are substantially the same as those set forth herein for skin tissue. Polynucleotides may be delivered to mucosal tissue and cells, or cells underlying the mucosa by injecting polynucleotide in naked, formulated or modified form into the mucosa, followed by electroporation with a noninvasive surface electrode, such as a caliper or meander electrode, known to those skilled in the art. Surface electrodes may be configured to fit the site of intended application, e.g. hollow organs or cavities. Alternatively, minimally invasive electrodes can be used, such as electrodes consisting of multiple, short-needle electrodes (U.S. Pat. No. 5,810,762; Glasspool-Malone, J., et al. Efficient nonviral cutaneous transfection. Molecular Therapy 2:140-146 (2000)), or saw tooth electrodes. Saw tooth electrodes are shaped as the name implies and can be applied in parallel rows of alternating polarities, with the tips of the teeth of the electrode penetrating deeper in the mucosa than the upper, wider portions of the saw teeth. The particles may also be injected into the mucosa by hollow needle or by fluid injection, or may be introduced by ballistic methods. One of skill can perform straightforward experiments to determine the optimal conditions for delivery of a DNA vaccine to a specific mucosal tissue.

The methods of the invention provide for cell-mediated immunity, and/or humoral or antibody responses. Thus, in addition to a conventional antibody response, the system herein described can provide, for, e.g., the association of the expressed antigens with class I MHC molecules such that an in vivo cellular immune response to the antigen of interest can be mounted including the production of CTLs to allow for future recognition of the antigen on target cells. Furthermore, the methods may elicit an antigen-specific response by helper T-cells. Accordingly, the methods of the present invention will find use with any antigen for which cellular and/or humoral immune responses are desired, including antigens derived from viral, bacterial, fungal and parasitic pathogens that may induce antibodies, T-cell helper epitopes and T-cell cytotoxic epitopes. Such antigens include, but are not limited to, those encoded by human and animal viruses and those expressed in heightened amounts on the surface of tumor cells, and can correspond to either structural or non-structural proteins.

If introduced separately from the polynucleotide vaccine into a tissue of the subject, the adjuvant particles are delivered to substantially the same site of delivery as the polynucleotide vaccine. The adjuvant particles can also be mixed with the polynucleotide vaccine for simultaneous delivery to the same site. Preferably, the DNA vaccine is mixed with 1×PBS or water and then the particles are added. In this embodiment, the particles are negatively or neutrally charged. Because the DNA is in solution, the particles and DNA do not chemically associate to any substantial extent.

The polynucleotide encoding an antigen and the particles (or formulations containing such agents) used in practice of the invention methods are introduced subcutaneously, generally by needle injection or by needle-free injection using a needle-free pressure-assisted injection system such asone that provides a small stream or jet with such force (usually provided by expansion of a compressed gas such as carbon dioxide through a micro-orifice within a fraction of a second) that the agent pierces the surface of the tissue and enters underlying dermal tissue, mucosa and/or muscle. The formulations can be injected mucosally, intradermally, subcutaneously, or intramuscularly, but are not applied to the surface of the skin (e.g., as topical solution, cream or lotion).

The invention methods can be used for inducing an immune response against any antigen whose nucleotide sequence is known and which causes disease in humans, and other mammals. For example antigens for several pathogenic intracellular viruses, such as those from the herpesvirus family are known, including those contained in proteins derived from herpes simplex virus (HSV)-types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpesviruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV.)

Polynucleotides encoding antigens from the hepatitis family of viruses, including hepatitis A virus (HAV), hepatitis B virus (HBV), hepatitis C virus (HCV), the delta hepatitis virus (HDV), hepatitis E virus (HEV), and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Polynucleotides encoding each of these proteins, as well as antigenic fragments thereof, will find use in the present methods.

Polynucleotides encoding antigens derived from other viruses will also find use in the claimed methods, such as without limitation, proteins from members of the families Picomaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1(also known as HTLV-III, LAV, ARV, hTLR, etc.)), including but not limited to antigens from the isolates HIV_{IIIb}, HIV_{SF2}, HIV_{LAV}, HIV_{LA1}, HIV_{MN}); HIV-1_{cM235}, HIV-1_{US4}; HIV-2; simian immunodeficiency virus (SIV) among others. Additionally, antigens may also be derived from human papillomavirus (HPV) and the tick-born encephalitis viruses. See, e.g. Virology, 3rd Edition (W. K. Joklikl ed. 1988); Fundamental Virology, 2nd Edition (B. N. Fields and D. M. Knipe, eds. 1991), for a description of these and other viruses.

More particularly, the gp120 envelope proteins any of thee above HIV isolates, including members of the various genetic subtypes of HIV, are known and reported (see, e.g., Myers et al., Los Alamos Database, Los Alamos National Laboratory, Los. Alamos, N.M. (1992); Myers et al., Human Retroviruses and Aids, 1990, Los Alamos, N.M.: Los Alamos National Laboratory; and Modrow et al., J. Virol. (1987) 61:570-578, for a comparison of the envelope sequences of a variety of HIV isolates) and antigens derived from any of these isolates alp will find use in the present methods.

Influenza virus is another example of a virus for which the present invention will be particularly useful. Specifically, the envelope glycoproteins HA and NA of influenza A are of particular interest for generating an immune response. Numerous HA subtypes of influenza A have been identified (Kawaoka et al., Virology (1990) 179:759-767, Webster et al., “Antigenic variation among type A influenza virses,” p. 127-168. In: P. Palese and D. W. Kingsbury (ed.), Genetics of influenza viruses. Springer-Veflag, N.Y.) Thus, proteins derived from any of these isolates can also be used in the immunization techniques described herein.

The methods described herein will also find use against numerous bacterial antigens, such as those derived from organisms that cause diphtheria, cholera, tuberculosis, tetanus, pertussis, meningitis, and other pathogenic states, including, without limitation, Meningococcus A, B and C, Hemophilus influenza type B (HIB), and Helicobacter pylori. Examples of parasitic antigens include those derived from organisms causing malaria and Lyme disease.

Furthermore, the methods described herein provide a means for treating a variety of malignant cancers. For example, the invention methods can be used to mount both humoral and cell-mediated immune responses to particular proteins specific to the cancer in question, such as an activated oncogene, a fetal antigen, or an activation marker. Such tumor antigens include, without limitation, any of the various MAGEs (melanoma associated antigen E), including MAGE 1, 2, 3, 4, etc. (Boon, T. Scientific American (March 1993):82-89); any of the various tyrosinases; MART 1 (melanoma antigen recognized by T cells), mutant ras; mutant p53; p97 melanoma antigen; CEA (carcinoembryonic antigen), among others. It is readily apparent that the subject invention can be used to prevent or treat a wide variety of diseases.

Dosage treatment may be a single dose schedule or a multiple dose schedule. A multiple dose schedule is one in which a primary course of vaccination may be with a single dose followed by other doses given at subsequent spaced time intervals, chosen to maintain and/or reinforce the immune response, for example at 4 weeks post primary vaccination for a second dose, and if needed, a subsequent dose after several weeks, for example up to 6 months post primary vaccination. The booster dose may be administered using the same type of particles, nucleotide-containing composition, and pulsed electric field as used to induce the primary immune response, or maybe administered and/or introduced using a different formulation or combination of immunization steps. Table 2 below illustrates the various combinations of treatment steps that can be used in the practice of the invention methods:

TABLE 2

| Method | Prime | Boost 1 | Boost 2 |
|---|---|---|---|
| 1 | DNA/particle | DNA/particle | DNA/particle |
| 2 | DNA/particle | DNA/particle | DNA |
| 3 | DNA/particle | DNA | DNA |
| 4 | DNA | DNA/particle | DNA/particle |
| 5 | DNA | DNA/particle | DNA |
| 6 | DNA/particle | DNA/particle | Protein |
| 7 | DNA/particle | DNA | Protein |
| 8 | DNA | DNA/particle | Protein |
| 9 | DNA/particle | DNA/particle | Protein/particle |
| 10 | DNA/particle | DNA | Protein/particle |
| 11 | DNA | DNA/particle | Protein/particle |
| 12 | DNA | DNA | Protein/particle |
| 13 | DNA/particle | Protein | Protein |
| 14 | DNA/particle | Protein/particle | Protein |
| 15 | DNA/particle | Protein/particle | Protein/particle |
| 16 | DNA | Protein/particle | Protein/particle |
| 17 | DNA | Protein/particle | Protein |
| 18 | DNA | Protein | Protein/particle |
| 19 | Protein/particle | Protein/particle | Protein/particle |
| 20 | Protein/particle | Protein | Protein |
| 21 | Protein/particle | Protein/particle | Protein |
| 22 | Protein | Protein/particle | Protein/particle |
| 23 | Protein | Protein/particle | Protein |
| 24 | Protein | Protein | Protein/particle |

The dosage regimen will also be determined, at least in part, by the need of the subject and be dependent on the judgment of the practitioner. Furthermore, if prevention of disease is desired, the invention methods are generally administered prior to primary infection with the pathogen of interest. If treatment is desired, e.g., the reduction of symptoms or recurrences, the invention methods are generally administered subsequent to primary infection.

The compositions will generally include one or more “pharmaceutically acceptable excipients or vehicles” such as water, saline, glycerol, polyethylene glycol, hyaluronic acid, ethanol, etc. Additionally, auxiliary substances, such as wetting or emulsifying agents, pH buffering substances, and the like, may be present in such vehicles.

Particles suitable for use in the present invention can also be derived, for example, from a poly α-hydroxy acid such as a poly(lactide) (“PLA”) or a copolymer of D,L-lactide and glycolide or glycolic acid, such as a poly (D,L,-lactide-co-glycolide) (“PLG” or “PLGA”), or a copolymer of D,L-lactide and caprolactone. The particles may be derived from any of various monomeric starting materials which have a variety of molecular weights and, in the case of the copolymers such as PLG, a variety of lactide:glycolide ratios, the selection of which will be largely a matter of choice, depending in part on the coadministered polynucleotide or polynucleotide-containing composition.

Alternatively, when the particles are liposomes (e.g., oil in water emulsions), the particles are derived from such vesicle-forming lipids as amphipathic lipids, which have hydrophobic and polar head group moieties and which (a) can form spontaneously into bilayer vesicles in water, as exemplified by phospholipids, or (b) are stably incorporated into lipid bilayers, with the hydrophobic moiety in contact with the interior, hydrophobic region of the bilayer membrane, and the polar head group moiety oriented toward the exterior, polar surface of the membrane. Although any type of liposome that is uncharged or negatively charged and which falls within the desired mean size range of 0.2 to 2 microns can be used, preferred types of liposomes are unilamellar and multilamellar liposomes.

The vesicle-forming lipids of this type typically include one or two hydrophobic acyl hydrocarbon chains or a steroid group and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Other vesicle-forming lipids include glycolipids, such as cerebrosides and gangliosides, and sterols, such as cholesterol.

Biodegradable polymers for manufacturing microparticles useful in the present invention are readily commercially available from, e.g., Boehringer Ingelheim, Germany and Birmingham Polymers Inc., Birmingham, Ala. For example, useful polymers for forming the particles herein include those derived from polyhydroxybutyric acid; polycaprolactone; polyorthoester; polyanhydride; as well as a poly(α-hydroxy acid), such as poly(L-lactide), poly(D,L-lactide) (both known as “PLA” herein), poly(hydoxybutyrate), copolymers of D,L-lactide and glycolide, such as poly(D,L-lactide-co-glycolide) (designated as “PLG” or “PLGA” herein) or a copolymer of D,L-lactide and caprolactone. Particularly preferred polymers for use herein are PLA and PLG polymers. These polymers are available in a variety of molecular weights, and the appropriate molecular weight for a given application is readily determined by one of skill in the art. Thus, e.g., for PLA, a suitable molecular weight will be on the order of about 2000 to 250,000. For PLG suitable molecular weights will generally range from about 10,000 to about 200,000, preferably about 15,000 to about 150,000, and most preferably about 50,000 to about 100,000.

If a copolymer such as PLG is used to form the particles, a variety of lactide:glycolide ratios will find use herein and the ratio is largely a matter of choice, depending in part on the coadministered polynucleotide or polynucleotide-containing vector or plasmid and the rate of degradation desired. For example, a 50:50 PLG polymer, containing 50% D,L-lactide and 50% glycolide, will provide a fast resorbing copolymer while 75:25 PLG degrades more slowly, and 85.15 and 90:10, even more slowly, due to the increased lactide component. Moreover, mixtures of microparticles with varying lactide:glycolide ratios will find use in the formulations in order to achieve the desired release kinetics for a given antigen and to provide for both a primary and secondary immune response.

The particles are prepared using any of several methods well known in the art. For example, double emulsion/solvent-evaporation techniques, such as described in U.S. Pat. No. 3,523,907 and Ogawa et al., Chem. Pharm. Bull. (1988) 36:1095-1103, can be used herein to form the particles. These techniques involve the formation of a primary emulsion consisting of droplets of polymer solution, which is subsequently mixed with a continuous aqueous phase containing a particle stabilizer/surfactant.

More particularly a water-in-oil-in-water (w/o/w) solvent evaporation system can be used to form the particles, as described by O'Hagan et al., Vaccine (1993) 11:965-969 and Jeffery et al., Pharm. Res. (1993)10:362. In this technique, the particular polymer is combined with an organic solvent such as ethyl acetate, dimethylchloride (also called methylene chloride and dichloromethane), acetonitrile, acetone, chloroform, and the like. The polymer will be provided in about a 2-15%, more preferably about a 4-10% and most preferably, a 6% solution, in organic solvent. An aqueous solution is added and the polymer/aqueous solution and emulsified using e.g., a homogenizer. The emulsion is then combined with a larger volume of an aqueous solution of an emulsion stabilizer such as polyvinyl alcohol (PVA) or, polyvinyl pyrrolidone. The emulsion stabilizer is typically provided in about a 2-15% solution, more typically about a 4-10% solution. The mixture is then homogenized to produce a stable w/o/w double emulsion. Organic solvents are then evaporated.

Oil-in water emulsions, such as liposomes, for use herein include nontoxic, metabolizable oils and commercial emulsifiers. Examples of nontoxic, metabolizable oils include, without limitation, vegetable oils, fish oils, animal oils or synthetically prepared oils. Fish oils, such as cod liver oil, shark liver oils and whale oils, are preferred, with squalene, 2, 6, 10, 15, 19, 23-hexamethyl-2,6,10,14,18,22-tetracosahexaene, found in shark liver oil, particularly preferred. The oil component will be present in an amount of from about 0.5% to about 20% by volume, preferably in an amount up to about 15%, more preferably in an amount of from about 1% to about 12% and most preferably from 1% to about 4% oil.

The aqueous portion of the particle adjuvant can be buffered saline or unadulterated water. If saline is used rather than water, it is preferable to buffer the saline in order to maintain a pH in the physiological range. Also, in certain instances, it may be necessary to maintain the pH at a particular level in order to insure the stability of certain composition components. Thus, the pH of the compositions will generally be pH 6-8 and pH can be maintained using any physiologically acceptable buffer, such as phosphate, acetate, tris, bicarbonate or carbonate buffers, or the like. The quantity of the aqueous agent present will generally be the amount necessary to bring the composition to the desired final volume.

Emulsifying agents suitable for use in the oil-in-water formulations include, without limitation, sorbitan-based non-ionic surfactants such as those commercially available under the name of SPAN® or ARLACEL® surfactants; polyoxyethylene sorbitan monoesters and polyoxyethylene sorbitan triesters, commercially known by the name TWEEN® surfactant; polyoxyethylene fatty acids available under the name MYRJ® surfactant; polyoxyethylene fatty-acid ethers derived from lauryl, acetyl, stearyl and oleyl alcohols, such as those known by the name of BRIJ® surfactant; and the like. These emulsifying agents may be used alone or in combination. The emulsifying agent will usually be present in an amount of 0.02% to about 2.50/by weight (w/w), preferably 0.05% to about 1%, and most-preferably 0.0% to about 0.5. The amount present will generally be about, 20-30% of the weight of the oil used.

The emulsions can also optionally contain other immunostimulating agents, such as muramyl peptides, including, but not limited to, N-acetyl-muramyl-L-threonyl-D-isoglutamine(thr-MDP), N-acteyl-normuramyl-L-alanyl-D-isogluatme (nor-DP), N-acetylmuramyl-L-alanyl-D-isogluatminyl-2-(1′-2′-dipalmitoyl-sn-glycero-3-huydroxyphosphoryloxy)-ethylamine (MTP-PE), etc. Immunostimulating bacterial cell wall components, such as monophosphorylipid A (MPL), trehalose dimycolate (TDM), and cell wall skeleton (CWS), may also be present.

For a description of methods of making various suitable oil-in-water emulsion formulations for use with the present invention, see, e.g., International Publication No. WO 90/14837; Remington: The Science and Practice of Pharmacy, Mack Publishing Company, Easton, Pa., 19th edition, 1995, Van Nest et al., “Advanced adjuvant formulations for use with recombinant subunit vaccines,”; In Vaccines 92, Modern Approaches to New Vaccines (Brown-et al., ed.) Cold Spring Harbor Laboratory Press, pp. 57-62 (1992); and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York (1995) pp. 277-296.

In order to produce particles less than 1 micron in diameter, a number of techniques can be used. For example, commercial emulsifiers can be used that operate by the principle of high shear forces developed by forcing fluids through small apertures under high pressure. Examples of commercial emulsifiers include, without limitation, Model 110Y microfluidizer (Microfluidics, Newton, Mass.), Gaulin Model 30CD (Gaulin Inc., Everett, Mass.), and Rainnie Minilab Type 8.30H (Miro Atomizer Food, and Dairy Inc., Hudson, Wis.). The appropriate pressure for use with an individual emulsifier is readily determined by one of skill in the art.

Particle size can be determined by, e.g., laser light scattering, using for example, a spectrometer incorporating a helium-neon laser. Generally, particle size is determined at room temperature and involves multiple analyses of the sample in question (e.g., 5×10 times) to yield an average value for the particle diameter. Particle size is also readily determined using scanning electron microscopy (SEM), photon correlation spectroscopy, and/or laser diffractometry. Particles for use herein will be formed from materials that are inert, sterilizable, non-toxic and preferably biodegradable.

The following are examples of specific embodiments for carrying out the present invention. The examples are offered for illustrative purposes only, and are not intended to limit the scope of the present invention in any way.

### EXAMPLE 1

Experiments were conducted to determine the level of transgene expression of DNA encoding secreted embryonic alkaline phosphatase (SEAP) in mice via electroporation-enhanced delivery of the DNA with or without the presence of particles that were not chemically associated with the DNA. In the first cohort, plasmid pSEAP-2 Control (Clontech Laboratories, Inc., Catalog #6052-1) (GenBank Accession Number U89938), which contains DNA encoding SEAP antigen mixed with 1×PBS was injected at a dosage of 5 μg in 50 μl into tibialis muscle of both legs of hairless mice (n=5).

In the second cohort of five hairless mice, DNA was administered using the same technique as for the first cohort and then electroporation was administered at substantially the same time, which, in this case, was immediately after DNA injection using a two needle electrode with needle spacing of 0.5 cm and the following electrical parameters provided by a ECM830 pulse generator (Genetronics): 6 pulses of 50V, for 20 Ms duration, 5 Hz;
In the third cohort of five hairless mice, DNA was administered using the same technique as for the first cohort along with adjuvant gold particles that were not chemically associated with the DNA. The particles had a size of 1.6 μm in diameter. The gold particles were weighed out (0.5 mg per injection site) and then combined with the DNA solution prepared in 1×PBS. The DNA and particles were mixed together well prior to injection.

In the fourth cohort of five hairless mice, DNA, electroporation and gold particles were administered using the same technique as described above, but with the electroporation being administered within 10-30 sec after injection.

Gene expression was measured in mouse sera using a SEAP reporter gene assay kit (Roche).

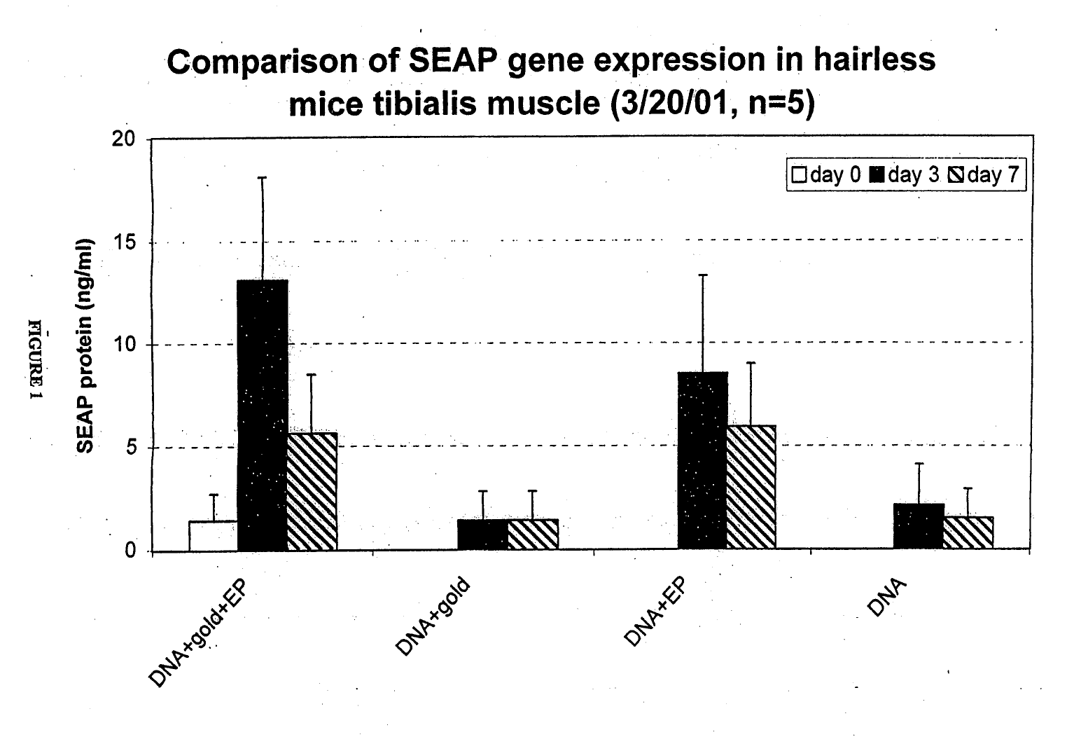

The results of these experiments are summarized in Table 3 below and in FIG. 1 in graphical form and show that the combination of adjuvant particles and EP results in a higher level of measurable gene expression as compared to injection of DNA alone or injection of DNA and particles, both without electroporation. In addition, the level of gene expression measured at days 3 and 7 in mice receiving the combination of adjuvant particles and EP is comparable or higher than that measured at days 3 and 7 in mice receiving DNA and EP without the particle adjuvant.

TABLE 3

| | Day 0 | | Day 3 | | Day 7 | |
|---|---|---|---|---|---|---|
| | Mean | St | Mean | St | Mean | St |
| | ng/ml | error | ng/ml | error | ng/ml | error |
| DNA + particle + EP | 1.4 | 1.3 | 13.1 | 5.0 | 5.6 | 2.9 |
| DNA + particle | | | 1.4 | 1.4 | 1.4 | 1.4 |
| DNA + EP | | | 8.5 | 4.8 | 5.9 | 3.1 |
| DNA | | | 2.1 | 2.0 | 1.5 | 1.4 |

t-test p-value between groups of “DNA + particle + EP” and “DNA + EP”: independent (0.32), paired (0.467).
St = Standard
“ng/ml” means ng of SEAP antigen per ml of blood serum

### EXAMPLE 2

### Influence of Particles on Immune Response after Electroporation Enhanced DNA Vaccination.

Further tests were conducted (1) to determine whether administration of adjuvant particles that are not chemically associated with the DNA vaccine has an additive effect or more than an additive effect on immune response generated by electroporation-enhanced administration of DNA vaccines, and (2) to compare if different target tissues (skin and muscle) produce different immune responses.

Two targeted tissues were selected: muscle and skin. For each target tissue, DNA vaccination was given to four cohorts of mice (see Table 4 below). Gold particles were administered with DNA concurrently by intramuscular or intradermal injection followed by electrporation; the gold particles and DNA were not chemically associated. Mice were primed, and then boosted twice, at week 4 and week 8 post-inmunization, respectively. Sera were tested for antibodies against specific antigen encoded by the vaccine DNA at week 2, 4, 6, 8 and 10; both primary and secondary immune antibody responses were evaluated.

TABLE 4

| Cohort | Target tissue | Treatment (2 sites per mouse) |
|---|---|---|
| 1 (control) | Muscle (i.m.) | DNA |
| 2 | ″ | DNA + EP |
| 3 | ″ | DNA + particle |
| 4 | ″ | DNA + particle + EP |
| 5 (control) | Skin (i.d.) | DNA |
| 6 | ″ | DNA + EP |
| 7 | ″ | DNA + particle |
| 8 | ″ | DNA + particle + EP |

Material and Methods

Mice: Balb/c, cohort size: 6 mice

DNA: ElsAg-expression vector encoding for the hepatitis B virus surface antigen (HbsAg). To generate the HbsAg expression construct, a 1.4 kb BamHI fragment of pAMS (ATCC) was inserted into pEF-BOS, an eukaryotic expression vector containing the human elongation factor 1α promoter and first intron and the polyadenylation signal from human. G-CSF cDNA in a Puc119 prokaryotic backbone (S. Mizushiama et al., Nucleic Acids Research 18:5322, 1990. pAM6(ATTC Nb. 45020) is a genomic clone of HBV serotype adw and the 1.4 kb BamHi fragment was shown to encode the “small” HBV surface antigen (HbsAG) (A. M. Moriarty et al., Proc. Natl. Acad. Sci. (USA) 78:2606-2620, 1981).

For immunization, each mouse was administered 10 μg of DNA in 50 μl PBS per site at two sites (tibialis muscle), or 10 μg of DNA in 25 μl PBS (skin site). Gold particles were mixed with the DNA, but not chemically associated with DNA, and were injected along with the DNA. Approximately 0.5 mg of particle were adminstered per injection site.

Assay: (1) ABBOTT AUSAB EIA with quantification panel to determine antibodies to HbsAg in Iu/mil. (2) anti-HbsAg ELISA to determine the endpoint antibody titers

Particles: BioRad Biolistic 1.6 Micron Gold Catalog Number: 1652264

Site and mode of immunization: (11) For intramuscular injections the site of injection was tibialis anterior muscles of both hind legs, (2). For intradermal injections, the site of injection was two sites on the dorsal skin on the lower back, by needle and syringe. Using the same protocol as the initial or prime immunization, the first and second boost were administered at weeks 4 and 8, respectively.

Electroporation conditions: (1) For intramuscular injections, electroporation was applied to tibialis muscle-using a Genetronics 2 needle array electrode with 5 mm needle distance with electrical pulses supplied by an ECM-830 pluse generator using the following settings: 50V, 20 msec., 6 pulses at 5 Hz. (2) For intradermal injections, electroporation was applied to dorsal skin using Genetronics meander electrodes. (width of electrode is 1 mm) with insulation (0.2 mm) between the electrodes with electrical pulses supplied by an ECM 830 pulse generator using the following settings: 70V, 20 ms, 3 pulses at 5 Hz.

Results: Table 5 below shows the results of ELISAs determining anti-HbsAg antibody endpoint titers for intramuscular (i.m.) and intradermal (id.) administration of polynucleotide and particles:

TABLE 5

| | | Secondary Response |
|---|---|---|
| | Primary Response | Week 6 Titer |
| Cohort | Week 4 Titer | (2 weeks post-booster 1) |
| i.m. DNA | 1:5000 | 1:2500 |
| | 1:1000 | 1:2500 |
| | 1:5000 | 1:2500 |
| | 1:5000 | 1:2500 |
| | 1:1000 | 1:2500 |
| | 1:1000 | 1:2500 |
| i.m. DNA + EP | 1:1000 | 1:50,000 |
| | >1:5000 | 1:50,000 |
| | 1:5000 | 1:25,000 |
| | 1:5000 | 1:25,000 |
| | 1:5000 | 1:25,000 |
| | >1:1000 | 1:2500 |
| i.m. | >1:5000 | 1:2500 |
| DNA + particle | 1:5000 | 1:2500 |
| | 1:5000 | 1:2500 |
| | 1:5000 | 1:2500 |
| | 1:5000 | 1:2500 |
| | >1:25 | 1:2500 |
| i.m. | 1:1000 | 1:2500 |
| DNA + particle + EP | >1:5000 | >1:50,000 |
| | >1:5000 | >1:50,000 |
| | >1:5000 | >1:50,000 |
| | >1:5000 | >1:50,000 |
| | >1:5000 | >1:50,000 |
| i.d. DNA | 1:1000 | 1:2500 |
| | 1:1000 | 1:2500 |
| | 1:1000 | 1:2500 |
| | 1:1000 | 1:2500 |
| | 1:1000 | 1:2500 |
| | 1:1000 | 1:2500 |
| i.d. DNA + EP | 1:1000 | 1:2500 |
| | 1:1000 | 1:2500 |
| | 1:5000 | >1:50,000 |
| | 1:1000 | 1:2500 |
| | >1:5000 | >1:50,000 |
| | >1:250 | 1:2500 |
| i.d. | >1:5000 | 1:2500 |
| DNA + pargicle | 1:1000 | 1:2500 |
| | 1:1000 | 1:25,000 |
| | 1:1000 | 1:2500 |
| | 1:1000 | 1:2500 |
| | 1:1000 | 1:250 |
| i.d. | >1:5000 | >1:50,000 |
| DNA + particle + EP | >1:5000 | 1:25,000 |
| | 1:5000 | >1:50,000 |
| | 1:5000 | 1:2500 |
| | 1:1000 | 1:25,000 |
| | 1:5000 | >1:50,000 |

Table 6 below shows the results of AUSYME EIAs determining Anti-HbsAg antibody titers for intramuscular (i.m.) and intradermal (i.d.) administration of polynucleotide and particles in mIU/ml (GMT).

TABLE 6

| | | | Secondary GMT | | |
|---|---|---|---|---|---|
| | Primary GMT | | (booster 1) | | booster 2 |
| cohort | Week 2 | Week 4 | Week 6 | Week 8 | Week 10 |
| DNA (i.m.) | 0 | 0 (0/6) | 0 (0/6) | 7 (1/6) | 40 (5/6) |
| DNA + EP | 1 | 10 (1/6) | 47 (5/6) | 129 (6/6) | 122 (6/6) |
| DNA + particle | 0 | 6 (2/6) | 13 (2/6) | 17 (4/6) | 65 (6/6) |
| DNA + | 4 | 15 (6/6) | 121 (5/6) | 130 (6/6) | 107 (6/6) |
| particle + EP | | | | | |
| DNA (i.d.) | 0 | 0 (0/6) | 0 (0/6) | 0 (0/6) | 0 (0/6) |
| DNA + EP | 0 | 2 (2/6) | 0 (4/6) | 88 (5/6) | 114 (6/6) |
| DNA + particle | 0 | 13 (1/6) | 0 (1/6) | 1 (2/6) | 14 (3/6) |
| DNA + | 1 | 18 (3/6) | 44 (5/6) | 82 (6/6) | 130 (6/6) |
| particle + EP | | | | | |

*GMT = Geometric mean titer calculated for responders. The number of responders per cohort, where applicable, is indicated in parenthesis.

Table 7 below shows the results of isotyping studies for cellular response for intramuscular (i.m.) and intradermal (i.d.) administration of polynucleotide and particles.

TABLE 7

| | | Secondary response | |
|---|---|---|---|
| | Primary response | (Booster 1) | (Booster 2) |
| Cohort | Week 4 | Week 6 | Week 10 |
| DNA (i.m.) | Th1-like, | | Th1-like, IgG2 |
| | IgG1 < IgG2 | | (3/3) |
| | ratio: 0.48 (1/3) | | |
| DNA + EP (i.m.) | Th1-like, | Th1-like, IgG1 < IgG2 | Th1/Th2 mixed, |
| | IgG1 < IgG2 | ratio: 0.22 (1/3) | IgG1 < IgG2 |
| | ratio: 0.31 (1/3) | | ratio: 0.30 (2/3), |
| | | | (IgG1 increased) |
| DNA + particle | Th1-like, | Th1-like, IgG2 (3/3) | Th1-like, |
| (i.m.) | IgG1 < IgG2 | | IgG1 < IgG2 |
| | ratio: 0.45 (1/3) | | ratio: 0.16 (1/3) |
| DNA + particle + EP | Th1-like, | Th1/Th2 mixed, | Th1/Th2 mixed, |
| (i.m.) | IgG1 << IgG2 | IgG1 < IgG2 ratio: 0 > 44 | IgG1 < IgG2 |
| | ratio: 0.18 (2/3) | (3/3), (IgG1 increased) | ratio: 0.40 (3/3), |
| | | | (IgG1, IgG2 |
| | | | increased) |
| DNA (i.d) | | | |
| DNA + EP (i.d.) | Th1/Th2 mixed | Th1-like, IgG1 < IgG2 | Th1/Th2 mixed, |
| | IgG1 < IgG2 | ratio: 0.26 (1/3) | IgG1 < IgG2 |
| | ratio: 0.46 (2/3) | | ratio: 0.24 (2/3), |
| | | | (IgG1 increased) |
| DNA + particle | | Th1-like, IgG2a (1/3) | Th1/Th2 mixed; |
| (i.d.) | | | IgG1 < IgG2 |
| | | | ratio: 0.43 (2/3), |
| | | | (IgG1 increased) |
| DNA + particle + EP | | Th1-like, IgG1 < IgG2 | Th1/Th2 mixed, |
| (i.d.) | | ratio: 0.19 (1/3) | IgG1 < IgG2 |
| | | | ratio: 0.26 (3/3), |
| | | | (IgG1, IgG2 |
| | | | increased) |

### Conclusions

The results of this study as summarized in Tables 5 and 6 show that the use of adjuvant particles that are not chemically associated with DNA vaccine enhances the immune response of electrically-assisted DNA vaccination. For example, the kinetics of the immune response following the invention method are faster than the other described methods, as shown by the strong antibody titers after primary immunization. Moreover, the quantity of the immune response his increased significantly earlier in the immune response with electroporation, similar titers were achieved with particle adjuvant after one boost as were achieved after two booster immunizations without particle adjuvant. The quality of the immune response (for example, the, appearance of Th1 response) is not altered by the presence of particle adjuvant: DNA vaccination causes predominant Th1 responses, as shown by the predominant IgG2 isotypes observed.

The combination of adjuvant particles, not chemically associated with the DNA vaccine, and, electrical assisted vaccine-delivery showed synergistic (better than additive) effect upon the immune responses after DNA vaccination in early phases (after the primary immunization and after a first booster dose).

### EXAMPLE 4

One way to measure the induction of cellular (Th1-type) responses after vaccination is to evaluate the level of protection afforded treated subjects when they are subsequently challenged with a tumor cell line expressing the antigen used for immunization. In immunized animals, antigen-modified tumor cells will be killed by CTLs, whereas unmodified tumor cells will not be seen by the immune system, allowing the outgrowth of tumor. Tumor challenge was performed by injecting immunized mice with CT26 cells, clone C12, which have been engineered to express HbsAg antigen by transfection with ElsAg expression vector (See Example 2 above). As a control, immunized mice were injected with an unmodified wild-type cell line (designated MDA). The results of the, tumor challenge tests are shown in Table 8 below.

TABLE 8

| | | | | Tumor Burden | | |
|---|---|---|---|---|---|---|
| | Target | | | post Challenge | | |
| Cohort | Tissue | Treatment | Challenge | Week 3 | Week 4 | Week 5 |
| 1 | Muscle | DNA | HBsAg | 0/3 | 1/3 | 2/3 |
| | (i.m.) | | | | | |
| | | | MDA | 2/3 | 3/3 | |
| | | | | | (sacrif.) | |
| 2 | Muscle | DNA + | HbsAg | 0/3/ | 0/3 | 0/3 |
| | (i.m.) | EP | MDA | 3/3 | | |
| | | | | (sacrif.) | | |
| 3 | Muscle | DNA + | HbsAg | 0/3 | 0/3 | 1/3 |
| | (i.m.) | particle | MDA | 3/3 | | |
| | | | | (sacrif.) | | |
| 4 | Muscle | DNA + | HBsAg | 0/3 | 0/3 | 1/3 |
| | (i.m.) | particle + | MDA | 3/3 | | |
| | | EP | | (sacrif.) | | |
| 5 | Skin | DNA | HBsAg | 1/3 | 1/3 | 1/3 |
| | (i.d.) | | | | | |
| | | | MDA | 3/3 | | |
| | | | | (sacrif.) | | |
| 6 | Skin | DNA + | HpsAg | 0/3 | 0/3 | 1/3 |
| | (i.d.) | EP | MDA | 3/3 | | |
| | | | | (sacrif.) | | |
| 7 | Skin | DNA + | HBsAg | 2/3 | 2/3 | 2/3 |
| | (i.d.) | particle | MDA | 3/3 | | |
| | | | | (sacrif.) | | |
| 8 | Skin | DNA + | HBsAg | 1/3 | 1/3 | 1/3 |
| | (i.d.) | particle + | MDA | 2/3 | 3/3 | |
| | | EP | | | (sacrif.) | |

The “tumor burden” depicts the number of animals showing any tumor growth at the indicated time points after administration of the CT26 cells. Because most of the animals were protected when challenged with the HBsAg-expressing cells, tumor antigen specific CTL cells are present and were induced by the DNA immunization protocol. When the same cell line was injected into the animals but the tumor antigen was not expressed, all but two animals succumb to tumor three weeks after challenge, with the remaining two animals not surviving one week later.

As shown by the data in Table 8, all modes of DNA vaccination generated sufficient cellular responses after primary immunization and two booster immunizations to produce substantial protection from challenge with a tumor cell line expressing the antigen used for the immunization. The tumorigenicity of the wild-type cell line (MDA) was demonstrated by fast and deadly tumor outgrowth. Thus, the invented method provides enhanced immunogenic effects without altering the desired cellular response.

### EXAMPLE 5

Further tests were conducted to determine whether administration of adjuvant particles would enhance immune responses when administered at various times after administration of the DNA vaccine and generation of the electric field. DNA vaccination and electroporation were administered cohorts of mice (n 10). Gold particles were administered to one cohort at the time of electroporation. A second cohort received the gold particles at day 1 after electroporation, a third cohort did not receive any particles. Mice were primed, sera were tested for vaccine-specific antibodies at week four, the time of the first booster immunization, and at week 6, two weeks after the booster immunization, to determine the secondary immune antibody response.

Mice: C57/B16 cohort size=10 mice.

DNA: ElsAg-expression, vector encoding the hepatitis B virus surface antigen (HbsAg) was administered using 25 μg of DNA in 50 μl of PBS per site. Gold was given at 1 mg per muscle, either mixed with the DNA but not chemically associated with it or in 50 μl of PBS for the day 1 cohort.

Assay: ABBOTT AUSAB EIA with quantification panel to determine antibodies to HbsAg in mIU/ml.

Particles: BioRad Biolistic 1.6 Micron Gold Catalog Number: 1652264

Site and mode of immunization: tibialis anterior muscles of both hind legs by needle and syringe.

Electroporation conditions: Genetronics 2 needle array etectrode with5 mm needle distance with electrical pulses supplied by an ECM 830 pluse generator using the following settings: 100V, 25 nmsec., 6 pulsesat 5 Hz.

The results of these tests, shown in Table 9 below, illustrate that particles, when mixed with DNA but not chemically associated with the DNA, and given at substantially the time of electroporation result in an enhanced immune response as compared to DNA vaccination and electroporation without particles. The greater enhancement was achieved when adjuvant particles were administered at the time of delivery of the DNA. When the adjuvant particles were administered one day after DNA transfer, there was still a measurable increase of immune response compared to mice that did not receive the adjuvant particles. In addition, this experiment showed that in low responder strains of mice, such as C57/B 16 mice used in this Example 5, particle adjuvant enabled production of an immune response for the dosage of DNA administered.

TABLE 9
GMT anti-HbsAg antibody titers in mIU/ml

| Cohort | Week 4 | Week 6 |
|---|---|---|
| DNA + EP with particles | 30.11 (9/10 positive) | 140.61 (10/10 positive |
| DNA + EP only | 1.86 (3/10 positive) | 1.25 (1/10 positive) |
| DNA + EP + particles | 7.59 (7/10 positive) | 10.68 (5/10 positive) |
| at day 1 | | |

Independent t-test of antibody titers after booster immunization:
DNA/EP with particles vs. DNA/EP only: p=0.0069
DNA/EP with particles vs DNA/EP, gold at day 1: p=0.059
DNA/EP with particles at day 1 vs. DNA/EP only: p=0.040

1. A method for inducing an immune response by administration of an antigen-encoding polynucleotide to a subject, said method comprising:
a) introducing an immunogenic-effective amount of a least one polynucleotide encoding an antigen into a target tissue of a subject by a route selected from the group consisting of, intramuscularly, intradermally, subcutaneously and intramucosally;
b) generating a pulsed electric field at the target tissue of sufficient strength and at substantially the same time as the introduction of the polynucleotide so as to result in the polynucleotide entering cells of the target tissue for expression therein and so as to result in generation in the subject of an immune response to the antigen encoded by the polynucleotide; and
c) introducing an adjuvant-effective quantity of particles into the target tissue within several days of the introduction of the polynucleotide and the generation of the electric field, wherein the polynucleotide and the particles are not substantially chemically associated with one another prior to the introducing thereof;
wherein the method enhances the immunogenicity of the polynucleotide encoding the antigen as compared with the immune response resulting from other modes of immunization involving administration of the polynucleotide encoding the antigen.

2. The method of claim 1, wherein the polynucleotide is introduced before the particles.

3. The method of claim 1, wherein the polynucleotide is introduced after the particles.

4. The method of claim 1, wherein the polynucleotide is introduced simultaneously with the particles.

5. The method of claim 1, wherein the immune response comprises a cellular immune response.

6. The method of claim 1, wherein the immune response comprises a humoral response.

7. The method of claim 1, wherein the immune response comprises generation of antibodies to the antigen encoded by the polynucleotide.

8. The method of claim 1, wherein the immune response is a T-cell mediated-immune response.

9. The method of claim 1, wherein the antigen is a tumor-associated antigen.

10. The method of claim 9, wherein the tumor-associated antigen is a cell-surface antigen.

11. The method of claim 10, wherein the tumor-associated antigen is a protein, polypeptide or polysaccharide.

12. The method of claim 1, wherein the polynucleotide is in a form selected from the group consisting of linear, relaxed, circular, supercoiled, condensed and chemically modified.

13. The method of claim 1, wherein the polynucleotide is DNA.

14. The method of claim 12, wherein the polynucleotide is contained in a vector or plasmid.

15. The method of claim 1, wherein the subject is mammal.

16. The method of claim 15, wherein the mammal is a human.

17. The method of claim 15, wherein the pulsed electric field is sufficient to cause electrotransport of the polynucleotide into cells of the tissue.

18. The method of claim 17, wherein the particles are selected from the group consisting of polymers, liposomes, microspheres and microparticles of biocompatible material.

19. The method of claim 18, wherein the particles are selected from the group consisting of particulate gold, aluminum, titanium, tungsten, and carbon.

20. The method of claim 19, wherein the pulsed electric field is generated in the target tissue by application of at least one electric pulse to at least two electrodes located in or on the surface of the tissue of the subject.

21. The method of claim 1, wherein the pulsed electric field is an electroporation-causing electric field.

22. The method of claim 21, wherein the pulsed electric field has a nominal electric field strength from about 50 V/cm to 400V/cm.

23. The method of claim 22, wherein the pulsed electric field has a nominal electric field strength from about 100 V/cm to 200 V/cm.

24. The method of claim 1, wherein the length of pulses in the pulsed electric field is from about 100 μsec to 100 msec.

25. The method of claim 1, wherein the waveform of the electric pulses is monopolar or bipolar.

26. The method of claim 1, wherein frequency of the pulses is from 0.1 to about 10 KHz.

27. The method of claim 1, wherein the particles are selected from the group consisting of microspheres and microparticles of biocompatible material.

28. The method of claim 27, wherein the particles are particulate gold or another noble metal.

29. The method of claim 27, wherein the particles are particulate titanium, tungsten, aluminum or carbon.

30. The method of claim 1, wherein the particles are polymers or liposomes.

31. The method of claim 1, wherein the particles have a largest mean dimension in the range from about 0.05 micron to about 20 microns.

32. The method of claim 31, wherein the particles have a largest mean dimension in the range from about 0.1 micron to about 3 microns.

33. The method of claim 1, wherein the pulsed electric field is generated in the target tissue by application of at least one electric pulse to at least two electrodes in or on the tissue of the subject.

34. The method of claim 1, wherein at least one electrode is inserted intradermally into the target tissue of the subject.

35. The method of claim 1, wherein the target tissue is skin and the electrodes are contained in a meander electrode.

36. The method of claim 1, wherein the target tissue is muscle and the electrodes are needle electrodes.

37. The method of claim 1, wherein the method is repeated at spaced intervals to administer booster dosages of the polynucleotide encoding the antigen or the antigen to the subject.

38. The method of claim 37, wherein the booster dosages are administered at one or more intervals selected from four weeks, 6 weeks, and 10 weeks after the initial administration.

40. The method of claim 1, wherein the polynucleotide encodes an antigen derived from a bacterial or viral pathogen.

41. The method of claim 1, wherein the particles are introduced up to three days before or after introduction of the polynucleotide and generation of the electric field.

42. A method for inducing an immune response by administration of antigen-encoding polynucleotide to a subject, said method comprising:
a) introducing an immunogenic-effective amount of at least one polynucleotide encoding an antigen into a target tissue of a subject by intramuscular injection;
b) generating a pulsed electric field at the target tissue of sufficient strength and at substantially the same time as the introduction of the polynucleotide so as to result in the polynucleotide entering cells of the target tissue for expression therein and so as to result in generation in the subject of an immune response to the antigen encoded by the polynucleotide; and
c) introducing an adjuvant-effective quantity of particles into the target tissue within several days of the introduction of the polynucleotide and the generation of the electric field, wherein the polynucleotide and the particles are not substantially chemically associated with one another prior to the introducing thereof;
wherein the method enhances the immunogenicity of the polynucleotide encoding the antigen as compared with the immune response resulting from other modes of immunization involving administration of the polynucleotide encoding the antigen.

43. The method of claim 42 wherein the particles are introduced up, to three days before or after introduction of the polynucleotide and generation of the electric field.

44. The method of claim 43, wherein the subject mammal.

45. The method of claim 44, wherein the mammal is a human.

46. The method of claim 44, wherein the pulsed electric field is sufficient to cause electrotransport of the polynucleotide into cells of the tissue.

47. The method of claim 46, wherein the particles are selected from the group consisting of polymers, liposomes microspheres and microparticles of biocompatible material.

48. The method of claim 47, wherein the particles are selected from the group consisting of particulate gold, aluminum, titanium, tungsten, and carbon.

49. The method of claim 48, wherein the pulsed electric field is generated in the target tissue by application of at least one electric pulse to at least two electrodes located in or on the muscle of the subject.
